(19) United States
(12) Patent Application Publication
(10) Pub. No.: US 2015/0038576 A1
(43) Pub. Date: Feb. 5, 2015

(54) COMPOSITIONS AND THEIR USE FOR SMOKING CESSATION AND OTHER TREATMENTS

(71) Applicant: SENTIENS, LLC, Charlotte, NC (US)

(72) Inventors: Inna S. Timokhina, Potomac, MD (US); Reid von Borstel, Potomac, MD (US); Dennis Tan, Weddington, NC (US); John Siverling, Matthews, NC (US)

(73) Assignee: SENTIENS, LLC, Charlotte, NC (US)

(21) Appl. No.: 14/449,533

(22) Filed: Aug. 1, 2014

Related U.S. Application Data
(60) Provisional application No. 61/861,865, filed on Aug. 2, 2013.

(51) Int. Cl. A24B 15/16; A61K 31/05; A24F 47/00; A61K 31/12; A61K 31/11; A61M 15/00; A61K 31/125; A61K 31/343

(52) U.S. Cl. CPC A24B 15/16; A61K 31/125; A61K 31/05; A61K 31/343; A61K 31/12; A61K 31/11; A61M 15/009; A24F 47/008
USPC 514/470; 131/270; 131/273; 514/692; 514/731; 514/678; 514/701; 128/200.14; 128/202.21

(57) ABSTRACT

A composition includes a liquid. The liquid aerosolizes for delivery to a user by an airway. The liquid includes an agent that activates a TRPV3 channel. The agent includes a terpenoid compound.

### CROSS-REFERENCE TO RELATED APPLICATIONS

This application claims priority to U.S. Provisional App. Ser. No. 61/861,865, filed Aug. 2, 2013, which is hereby incorporated by reference in its entirety.

### BACKGROUND

Chronic exposure to cigarette smoke can lead to inflammatory airway conditions, such as COPD, and tobacco smoking can cause cancer and respiratory and cardiovascular disease. These negative health effects can be attributed to the carcinogenic, oxidant and other toxic constituents of cigarette smoke. It has been estimated that up to 50% of heavy smokers develop chronic obstructive pulmonary disease (COPD), while prevalence of COPD in general population is estimated as 10%. Chronic obstructive pulmonary disease (COPD) is now recognized as a leading cause of morbidity and mortality worldwide and is associated with significant individual and socioeconomic burden. Furthermore, COPD patients can have high levels of anxiety and depression, which are related to progressive worsening of the disease. Exposure to tobacco smoke is a significant etiologic agent of this disease, which is characterized by progressive airflow limitation with an abnormal inflammatory response in the small airways and alveoli, small airway remodeling, chronic bronchitis, pulmonary hypertension and emphysema. The inflammation induced by cigarette smoke can lead to an increase in protease production, a major contributor to lung destruction, seen in emphysema. Other factors contributing to the development of COPD include exposure to dusts, fumes and air pollution particles.

### BRIEF SUMMARY

In an embodiment, a composition includes a liquid. The liquid aerosolizes for deliver to a user by an airway. The liquid includes an agent that activates a TRPV3 channel. The agent includes a terpenoid compound.

In another embodiment, a device includes a liquid and a housing. The liquid aerosolizes for deliver to a user by an airway. The liquid includes an agent that activates a TRPV3 channel. The agent includes a terpenoid compound. The housing delivers the liquid aerosolized to the user.

### BRIEF DESCRIPTION OF THE DRAWINGS

FIG. 1 is a perspective view of an exemplary device for delivering constituents of the present disclosure.

FIG. 2 is a perspective view of an exemplary inhaler device for delivering constituents of the present disclosure.

FIG. 3 is an exploded schematic side view of an exemplary device for delivering constituents of the present disclosure.

### DETAILED DESCRIPTION

Embodiments described herein relate generally to a composition, methods and devices for delivering the composition, that exhibit properties of the present disclosure.

The present disclosure is generally directed to compositions, apparatuses and methods for delivering compositions that act on transient receptor potential V3 (TRPV3) channels, expressed in sensory neurons of the respiratory tract. In some embodiments, an apparatus takes the form of a cigarette substitute device for alleviating cigarette craving or supporting smoking cessation. The cigarette substitute device may include such devices as electronic cigarettes, non-electronic cigarette substitute devices with or without heating elements, inhalers, vaporizers, and any other device that may deliver the composition to the airway.

In the following discussion, cigarette smoking is used as a generic and illustrative term to include cigarette, cigar or pipe smoking without loss of generality to smoking in general.

Embodiments of the disclosure provide compositions of bioactive plant derived compounds, which may be nicotine and tobacco free. Embodiments of the disclosure further provide methods and devices for delivering the compositions to the respiratory tracts of smokers, former smokers or other subjects, using vaporizing devices. The constituents of the compositions may possess anti-inflammatory, anti-oxidant and/or anti-anxiety activities. The compositions, methods and devices provide improved mimicry of smoking and suppress smoking abstinence symptoms.

Mechanisms of neurogenic and tissue inflammation in the respiratory tract induced by cigarette smoke will now be discussed. Chemical irritants are detected by chemosensitive C-fibers—e.g. sensory neurons that extend numerous terminals superficially into the airway epithelium, placing them in an ideal position to react to inhaled irritants. These neurons express chemosensory TRP receptors, including TRPA1, TRPV1, TRPV3 and TRPM8. The TRPA1 receptor is a broad spectrum sensor for irritants due to its structure, and it plays an important role in sensing noxious tobacco smoke constituents. Furthermore, TRPA1 mediates neurogenic inflammation of sensory nerves induced by unsaturated aldehyde components of cigarette smoke such as acrolein and crotonaldehyde. TRPA1 is also a sensor for multiple oxidants contained in cigarette smoke.

Transient sensitization of airway neurons by cigarette smoke or chemical irritants contributes to airway protection, eliminating these irritants from the respiratory tract and promoting tissue healing and recovery. In contrast, persistent neuronal sensitization by cigarette smoke in habitual smokers may lead to respiratory irritation and neurogenic inflammation in a TRPA1 dependent manner. Inflamed nerves secrete pro-inflammatory neuropeptides induced by toxic and oxidant components of cigarette smoke thereby further promoting tissue inflammation and damage. Thus, neuronal inflammation occurring in parallel with airway tissue inflammation can lead to development of inflammatory airway conditions, such as COPD and asthma.

Mechanisms of chronic conditions of itch and cough induced by cigarette smoke will now be discussed. Multiple TRPA1 activators, particularly cigarette smoke, evoke cough in animals and humans. TRPA1 may act as a mediator of histamine independent itch. Oxidants cause itch and resulting scratching behavior in a TRPA1 dependent manner. Oxidative stress is indicative of most acute and chronic inflammatory airway conditions. Reactive oxygen species (ROS) are generated by infiltrating macrophages and neutrophils during inflammation. Thus, itch and cough may be induced both by toxic and oxidant constituents of cigarette smoke and by endogenous inflammatory mediators released by inflamed tissues in the respiratory tract of heavy smokers. Continuous exposure of airway tissues to this “inflammatory soup” may provide a basis for progressive worsening of the disease, which may be observed in COPD and asthma, as well as reactive respiratory tract syndrome (RADS). These inflammatory airway conditions may be considered diseases of chemical sensing.

Despite well-known adverse health consequences of smoking, approximately 20% of U.S. adults smoke tobacco cigarettes. This number is higher in other countries. Quitting smoking is difficult because of the aversive withdrawal symptoms that accompany tobacco abstinence. Symptoms of tobacco abstinence include physical complaints such as headache and hunger, negative mood and high irritability, decrease in attention and cognitive function, and increased cigarette craving and smoking urges.

Inflammatory “throat itch” and the desire for “throat scratch” may be major contributors for urges to smoke and cravings for a cigarette (e.g., withdrawal symptoms). Toxic and oxidant components of cigarette smoke may induce neurogenic inflammation, tissue inflammation and itch. Itch (e.g., pruritus) is an unpleasant sensation that elicits the desire or reflex to scratch. Acute itch may serve as a warning and self-protective mechanism to protect a person from potentially harmful irritations. However, chronic itch may also be a common clinical problem that is associated with variety of diseases. Although itch sensation can be transiently relieved by scratching, itch-scratch cycles may exacerbate cutaneous problems and lead to further injury.

A component of smoking for relief of acute cigarette withdrawal symptoms is the sensation of tobacco smoke in the respiratory tract, particularly a throat and tracheal sensation known as “throat scratch” or “throat hit”. Consistent with this, denicotinized cigarettes may be moderately effective in suppressing some withdrawal symptoms, such as the urges to smoke, but not other symptoms attributable to dependence on nicotine, such as difficulty concentrating or increased eating. Furthermore, the craving for a cigarette may be a withdrawal symptom that could be suppressed by non-nicotine stimuli, such as citric acid inhaler. The degree of a smoker's satisfaction correlates with the intensity of the “throat scratch”.

Therefore, inflammatory “throat itch” and desire for “throat scratch” can be major contributors to smoking urges. Reducing inflammatory itch by delivering anti-oxidants and anti-inflammatory compounds to the respiratory tract of heavy smokers may therefore reduce inflammatory itch, desire for throat scratch and smoking urges.

Nicotine-dependent abstinence symptoms include anxiety and increased stress reactivity. One factor that contributes to tobacco abstinence symptoms is physical dependence on tobacco-delivered nicotine. Nicotine is recognized as an addictive psychoactive drug and an anxiolytic. Some individuals smoke to relieve anxiety. Stress can also lead to cigarette craving in abstinent humans, and, since tobacco withdrawal is itself stressful, it makes abstinent individuals even more vulnerable to life's other stresses. Nicotine withdrawal is associated with dysregulation of the hypothalamic pituitary adrenal axis and increased stress reactivity. Nicotine withdrawal may also be associated with dysfunction of the dopamine system. Experimental evidence in humans and animals indicates a role of dopamine in stress-induced reinstatement of drug taking (relapse). Furthermore, anxiety sensitivity is a trait associated with habitual smoking and is a predictor of acute subjective effects of smoking.

Anxiety disorders, as a group, are among the most common mental health conditions, frequently cause significant functional impairment and pose risk factors for numerous additional diseases. Thus, suppressing anxiety associated with withdrawal from smoking addiction may be an important factor for increasing smoke cessation rates and reducing negative side effects of withdrawal.

Nicotine replacement therapy medications can increase quit rates an average 1.4 times compared to placebo. However, even with the use of medications, the success rate of quitting remains low. The percentage of smokers who relapse within six months with the use of nicotine replacement therapies is reported to be 93%. Although many nicotine replacement therapies products have been available in the US during the past decade, the overall quit rate has changed very little, from 48.7% in 1998 to 51.1% in 2008. Furthermore, many smokers cite negative side effects of nicotine replacement therapies and their ineffectiveness in preventing relapse.

A variety of potential reduced exposure products have been marketed to cigarette smokers with explicit or implied claims that their use is associated with less exposure to toxic smoke constituents. Electronic cigarettes, or E-cigarettes, are one of the newer types of potential reduced exposure products available. E-cigarettes are battery-operated devices may vaporize a liquid solution of propylene glycol or vegetable glycerin in which nicotine and tobacco-like flavors may be dissolved. Puffing activates a battery-operated heating element in an atomizer and a liquid in a cartridge is vaporized as a mist that may be inhaled. The user thus receives a smoke-like vapor bearing nicotine without the full burden of toxic and carcinogenic tobacco combustion products.

E-cigarettes are often designed to look like traditional cigarettes and simulate the visual, sensory, and behavioral aspects of smoking traditional cigarettes. E-cigarettes may present a new way to facilitate successful smoking cessation since they address both biochemical and behavioral aspects of smoking addiction. At least for some smokers, electronic cigarettes may be able to replace tobacco cigarettes. However, e-cigarettes may not provide effective nicotine delivery and may also be ineffective at suppression of abstinence symptoms for many smokers.

E-cigarette devices have not previously offered effective nicotine addiction cessation being limited to a less harmful cigarette replacement tool that only partially suppresses abstinence symptoms. Thus, there is a need to develop devices that are more efficient in suppressing both nicotine and non-nicotine withdrawal symptoms in abstinent smokers to increase smoke cessation rates.

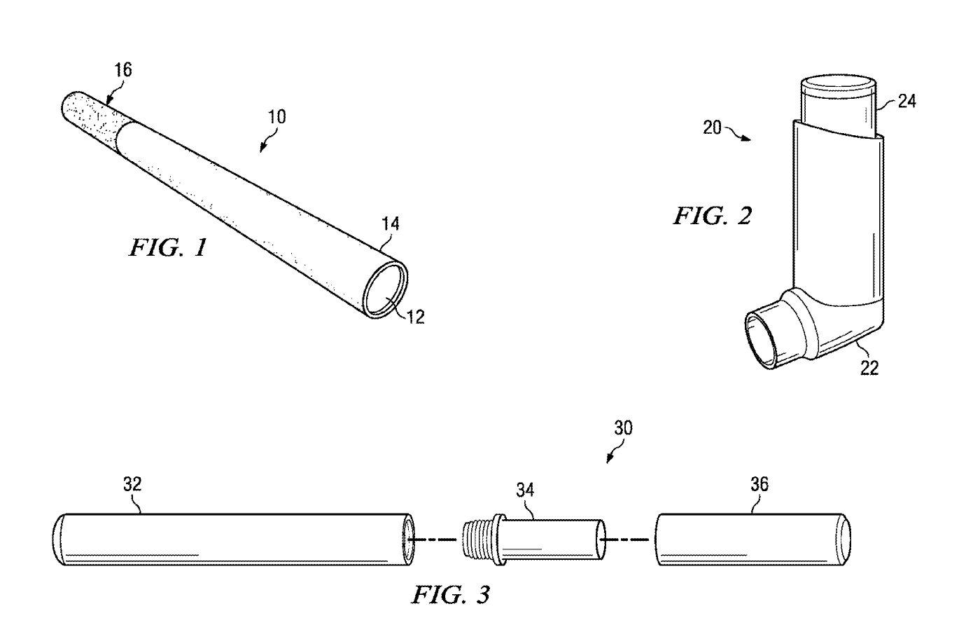

Referring to FIG. 1, in some embodiments, an exemplary cigarette substitute device 10 may be approximately the size and shape of a conventional cigarette. Volatile constituents may be dispersed within a porous matrix 12 encased within a relatively nonporous, liquid impermeable wrapper 14 resembling the outer wrap paper of a conventional cigarette. The device 10 is effective without use of applied heat or electrical or mechanical energy apart from suction applied by the user's inhalation at an end 16 of the device 10. The end 16 may be constructed to resemble a conventional filter rod in appearance and/or feel. In some embodiments, a filter rod is used at the end 16 of the device 10. The device 10, without heat, electrical power or aerosol generation, uses volatile active constituents, which in turn restrict the amount of material that can be delivered versus generated aerosols or smoke. Thus, cigarette-mimicking activity can be achieved. This principle is not restricted to cigarette-like devices, but also includes substitute cigars, pipes, and other devices used to smoke tobacco. For example, vaporizing devices which may not specifically resemble the physical appearance of a common tobacco smoking implement, but which serve the function of heating, via an electrical heating coil or other heating element such as a butane catalyst heater, a liquid or porous substrate containing a composition of the disclosure to volatilize or vaporize it for delivery to the respiratory tract via inhalation through the mouth or nose are also contemplated.

The principles of the present disclosure may further be applied to inhaler devices. Referring to FIG. 2, an inhaler device 20 includes a housing 22 and a canister 24. The active constituents are disposed as a solution in the canister 24. When the canister 24 is depressed into the housing 22, a portion of the active constituents are released into the housing 22 in a volatized form. Thus, when the user inhales from the housing 22, the user receives the volatized active constituents.

In one embodiment, compositions of the disclosure are delivered to the airway with an inhaler similar to those used for asthma medications or with a spray device similar to those used for breath fresheners.

The compositions of the present disclosure may also be provided as liquids used in electronic cigarettes or electric vaporizers in conjunction with either an absence or reduced levels of nicotine to provide an adequate simulation of the chemosensory experience of inhaling aerosols containing nicotine as an aid to reducing dependence upon, or craving for, nicotine. Referring to FIG. 3, an electronic cigarette device 30 includes a battery 32, an atomizer/vaporizer 34 and a cartridge 36. The active constituents are disposed as a solution in the cartridge 36. The atomizer 34 uses energy from the battery 32 to volatize the active constituents, which are then delivered to a user inhaling from an end of the device 30.

An object of this disclosure is to provide electronic cigarette liquid constituents that possess anti-inflammatory and anti-oxidant activities that relieve inflammatory itch in the respiratory tract and reduce smoking urges. In addition, these constituents may provide warming sensation in the respiratory tract and improve mimicry of cigarette smoke sensations. Furthermore, the constituents in embodiments of the compositions of this disclosure may provide anxiolytic activity to further support smoking cessation.

Embodiments of this disclosure include electronic cigarette liquids containing plant-derived compounds that act on one or more transient receptor potential V3 (TRPV3) channels expressed in sensory neurons of the respiratory tract to provide relief from smoking abstinence symptoms such as smoking urges and anxiety.

In an embodiment, a composition of bioactive plant derived TRPV3 activators may be delivered to the respiratory tract of smokers using electronic vaporizers. The constituents of this composition may include anti-inflammatory, anti-oxidant and anti-anxiety activities and provide improved mimicry of smoking to suppress smoking abstinence symptoms.

In an embodiment, a method of reducing cigarette craving includes inhibiting chronic inflammatory itch in smokers respiratory tract and associated desire for a throat scratch provided by the process of smoking.

In an embodiment, a method of reducing cigarette craving includes providing a warming sensation to mimic process of smoking.

In an embodiment, a method includes relieving anxiety and increased irritability associated with smoking abstinence to support smoking cessation.

TRPV3 is a class of TRP channels and is highly expressed in olfactory and respiratory epithelial cells and sensory nerves including those, by way of example, innervating the respiratory tract. TRPV3 may be activated by warm temperatures of a physiological range (e.g., 31-39 C) and elicits feeling of warmth, whether activated by elevated temperature or by chemical activators. Thus, when TRPV3 active constituents are inhaled in aerosol droplets (e.g., from an electronic cigarette device) the resulting sensation of warmth in the respiratory tract may provide improved mimicry of smoking.

There are relatively few chemical agonists for TRPV3 receptors. Chemical agonists for TRPV3 include terpenoid compounds of plant origin, including camphor (e.g., from Rosmarinus officinalis), carvacrol and thymol (e.g., from Thymus Vulgaris and Origanum vulgare), and incensole acetate (e.g., from Frankincense Boswellia sacra).

TRPV3 activators may exhibit anti-anxiolytic properties. Incensole acetate, a macrocyclic diterpenoid, derived from Boswellia resin is a potent agonist of TRV3 channel. Incensole acetate may elicit anti-anxiolytic and anti-depressive activities. These anti-anxiolytic effects may be mediated by a TRPV3 receptor. Incensole acetate may also influence hippocampal gene expression, which may lead to beneficial behavioral effects.

Phenolic monterpene carvacrol, a major constituent of extracts of Thymus Vulgaris and Origanum vulgare, may provide anti-depressive and anti-anxiolytic effects. Furthermore, extract of Origanum vulgare may elevate serotonin levels in the brain and positive behavioral effects in animal models. Carvacrol may be responsible for biological activities of Origanum vulgare.

TRPV3 activators may exhibit anti-inflammatory and anti-oxidant properties. Incensole acetate may elicit robust anti-inflammatory effects in vivo and in vitro and inhibit NF-kB activation. Thymol, as well as number of other terpenoid compounds, may elicit strong anti-inflammatory properties using Th1/Th2 cytokine secretion profiles using murine splenocytes. Carvacrol may also have anti-inflammatory and anti-microbial activities in vitro and in vivo. Essential oil of Rosmarinus Officinalis may elicit significant anti-inflammatory effects, and camphor may strongly inhibit pro-inflammatory cytokine release in relevant human cell systems.

Essential oil of Thymus Vulgaris, consisting mainly of thymol and carvacrol, may exhibit significant anti-oxidant activities. Furthermore, thymol and carvacrol may elicit very strong anti-oxidant activities, for example through inhibition of lipid peroxidation assays and free radical scavenging assays. These compounds may provide strong anti-oxidant activities as compared to a panel of biologically active plant-derived compounds.

Essential oil of Thymus Vulgaris, including mainly thymol and carvacrol, may exhibit significant anti-oxidant activities, for example through radical scavenging assays. Furthermore, thymol and carvacrol, as individual components, may elicit very strong anti-oxidant activities, as determined for example using inhibition of lipid peroxidation assays and free radical scavenging assays. These compounds may provide strong anti-oxidant activities as compared to a panel of biologically active plant-derived compounds.

### First Exemplary Embodiment

In a first exemplary embodiment, an e-cigarette liquid composition includes one or more plant derived TRPV3 activators, which are selected from camphor, thymol, carvacrol and incensol acetate. The constituents of this composition may possess anti-inflammatory, anti-oxidant and anti-anxiety activities, may provide improved mimicry of smoking and may suppress smoking abstinence symptoms, such as smoking urges and anxiety.

### Second Exemplary Embodiment

In a second exemplary embodiment, a method of reducing cigarette craving includes a step of administering the constituents from the first exemplary embodiment to the respiratory tract of smokers using an electronic vaporizer. This embodiment is particularly useful for smokers experiencing inflammatory airway conditions such as COPD due to anti-inflammatory and anti-oxidant activities that may be provided by these constituents. When administered over a course of several weeks the composition of the first exemplary embodiment may provide relief of inflammatory itch, desire for throat scratch and smoking urges. During the course of treatment, the inflammation of the respiratory tract may be monitored using, for example, clinical laboratory methods to analyze exhaled biomarkers such as volatile organic compounds (VOCs) and by conducting a questionnaire of smoking urges.

### Third Exemplary Embodiment

In a third exemplary embodiment, a method of reducing anxiety and irritability associated with smoke abstinence includes a step of administering the constituents from the first exemplary embodiment to the respiratory tract of smokers using electronic vaporizer. This embodiment is particularly useful for smokers, experiencing increased anxiety sensitivity and/or anxiety disorders due to inherent anxiolytic activities of these constituents. When administered over a course of several weeks the composition of embodiment 1 provides relief from anxiety, increased stress reactivity and irritability. During the course of treatment, the anxiety diagnoses may be monitored using, for example, a structured clinical interview for anxiety disorders or functional magnetic resonance imaging.

### Fourth Exemplary Embodiment

An unexpected property of TRPV3 active compounds of this disclosure is that they may reduce the pro-tussive effect of agents that activate TRPA1. In a fourth exemplary embodiment, an electronic cigarette liquid includes a TRPA1 activator such as 6-paradol or Cinnamomum Verum aldehyde to provide “throat scratch” sensation that is augmented with one or more plant derived TRPV3 activators, which are selected from camphor, thymol, carvacrol and incensol acetate. The strong throat sensation of the TRPA1 activator may be maintained and further improved by the diffuse warm sensation of the TRPV3 activator(s) with a reduction in the cough reflex otherwise elicited by the TRPA1 activator.

### Fifth Exemplary Embodiment

In a fifth exemplary embodiment, an electronic cigarette device includes a composition of the fourth exemplary embodiment, which may be useful for smokers experiencing throat itch and urges to smoke. This device may provide improved mimicry of smoking sensations including the sensation of warmth and “throat scratch” without the irritation characteristic to inhalation of cigarette smoke.

In another embodiment, a method of reducing cigarette craving includes a step of administering the constituents from the fourth exemplary embodiment to the respiratory tract of smokers using electronic vaporizer.

In another embodiment, a series of electronic cigarette liquids with progressively lower nicotine content is prepared. As nicotine is reduced in successive portions of liquid, TRPA1 and TRPV3 activators (such as those of the fifth exemplary composition) are added in quantities sufficient to maintain the same or greater intensity of throat sensation. The series of electronic cigarette liquids, which may be deployed in a corresponding series of electronic cigarette devices or supplied successively to a reloadable electronic cigarette device, may provide gradual weaning from nicotine by maintaining the sensory, tactile and behavioral elements with progressive reduction of nicotine.

In another embodiment, an electronic cigarette device may be used, not as a smoking cessation device or cigarette substitute, but as a delivery vehicle for therapeutic inhalation of botanical extracts or constituents that activate TRPV3. Such a device and composition may be useful for providing antitussive effects, bronchodilation, and relief of cold or asthma symptoms. In addition, via both sensory and post-absorptive effects, compositions and devices of this disclosure may provide relief from symptoms of anxiety and depression whether due to cessation of addictive behavior involving tobacco or to other causes. This device may be particularly useful for COPD patients since COPD may be associated with high levels of anxiety and depression.

In another embodiment, an electronic cigarette device includes a composition of the first exemplary embodiment, which may be useful for patients, post-exposure, to occupational hazards or environmental disasters such as vapors, gasses, dusts and/or fumes. Post-exposure treatment with this device may reduce sensory airway irritation and prevent or reduce adverse long-term health effects elicited by neurogenic inflammation.

In another embodiment, a method of reducing sensory airway irritation caused by exposure to occupational hazards or environmental disasters includes a step of administering the constituents from the first exemplary embodiment to the respiratory tract of these patients using electronic vaporizer.

In another embodiment, an electronic cigarette device containing a composition of the first exemplary embodiment may be used to relieve seasonal variations in the acute exacerbations of patients with COPD, asthma, or viral respiratory infections. Such a device and composition may be useful for providing antitussive effects, bronchodilation, and relief of cold or asthma symptoms.

In another embodiment, a method of reducing seasonal acute exacerbations of patients with COPD, asthma, or viral respiratory infections includes a step of administering the constituents from the first exemplary embodiment to the respiratory tract of these patients using an electronic vaporizer.

### First Exemplary Composition

200 grams of powdered dry leaves of Thymus Vulgaris were extracted with 1 liter of 95% ethanol by heating in a glass vessel suspended in 170 degree F. water while stirring. The supernatant was passed through filter paper and concentrated by distillation of the ethanol in a cold finger apparatus. The residue (approximately 20 ml) was made up to a volume of 40 ml with ethanol. The Thymus Vulgaris concentrate was used as a constituent of an electronic cigarette liquid using a base of propylene glycol. When an e-cigarette device containing Thymus Vulgaris concentrate is activated by inhalation, it provided a distinct pleasant and warming sensation.

### Second Exemplary Composition

200 grams of powdered dry leaves and flowers of Origanum Vulgare were extracted with 1 liter of 95% ethanol by heating in a glass vessel suspended in 170 degree F. water while stifling. The supernatant was passed through filter paper and concentrated by distillation of the ethanol in a cold finger apparatus. The residue (approximately 20 ml) was made up to a volume of 40 ml with ethanol. The Origanum Vulgare concentrate was used as a constituent of an electronic cigarette liquid using a base of propylene glycol. When an e-cigarette device containing Origanum Vulgare concentrate is activated by inhalation, it provided a distinct pleasant and warming sensation.

### Third Exemplary Composition

200 grams of powdered dry leaves of Rosmarinus officinalis were extracted with 1 liter of 95% ethanol by heating in a glass vessel suspended in 170 degree F. water while stirring. The supernatant was passed through filter paper and concentrated by distillation of the ethanol in a cold finger apparatus. The residue (approximately 20 ml) was made up to a volume of 40 ml with ethanol. The Rosmarinus Officinalis concentrate was used as a constituent of an electronic cigarette liquid using a base of propylene glycol. When e-cigarette an device containing Rosmarinus Officinalis concentrate is activated by inhalation, it provided a distinct pleasant and warming sensation.

### Fourth Exemplary Composition

Extracts of Aframomum melegueta seeds, Cinnamomum Verum, Thymus Vulgaris and Rosmarinus Officinalis were prepared as in the first exemplary composition and combined in the following proportions that can be varied: up to 15% aframomum melegueta; up to 15% Cinnamomum Verum; up to 70% Thymus Vulgaris; up to 15% rosmarinus officinalis.

When this mixture diluted with propylene glycol (or optionally with propylene glycol containing up to 20% ethanol) was vaporized in an electronic cigarette or similar device and inhaled, there were clearly identifiable effects of throat hit, sensation of warmth and calming effect, with an amelioration of the cough reflexes otherwise elicited by the TRPA1 activators derived from the aframomum melegueta and Cinnamomum Verum extract.

### Fifth Exemplary Composition

The following ingredients were pulverized in a grain mill and extracted in 1 liter of 95% ethanol by heating for two hours at reflux temperature: 30 to 90 g Aframomum melegueta seeds; 1 to 5 g Szechuan pepper; 1 to 4 g Cinnamomum Verum; 10 to 40 g Thymus Vulgaris; 5 to 20 g Eucalyptus leaves; and 1 to 20 g Rosmarinus officinalis leaves. The extract was filtered and evaporated down to a volume of 100 ml after addition of 0.1 ml of cedar absolute (1:1 dilution in ethanol), extracted with water to remove water-soluble constituents, and then evaporated to a residue.

When this residue was diluted with propylene glycol (or optionally with propylene glycol containing up to 20% glycerol) and then vaporized in an electronic cigarette or similar device and inhaled, there were clearly identifiable effects of throat hit, sensation of warmth and calming effect, with an amelioration of the cough reflexes otherwise elicited by the TRPA1 activators derived from the aframomum melegueta and Cinnamomum Verum extract.

It will be appreciated that the described exemplary botanical constituents are not limiting and the described constituents include equivalents such as synthetic alternatives. It will also be appreciated that description of constituents in compositions as by weight or by volume is merely exemplary and is not limiting. Constituents may be measured using any of a variety of available methods.

In addition, it will be appreciated that the above described devices and compositions are not limited to cigarette-like rods, but are also applicable to other devices such as inhalers that may be used to deliver the volatile constituent(s). Also, it will be appreciated that the above described devices are applicable to applications beyond smoking substitution and smoking cessation. As another example, devices and compositions contemplated herein can also be used for delivery of aromatic agents useful for clearing respiratory tract in people with colds.

While various embodiments in accordance with the disclosed principles have been described above, it should be understood that they have been presented by way of example only, and are not limiting. Thus, the breadth and scope of the invention(s) should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically and by way of example, a description of a technology in the “Background” is not to be construed as an admission that technology is prior art to any invention(s) in this disclosure. Neither is the “Summary” to be considered as a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

What is claimed is:

1. A composition for delivery to the respiratory tract of a subject, comprising:
a liquid including an agent, the agent being an activator of a TRPV3 channel, and the agent including a terpenoid compound.

2. The composition of claim 1, wherein the liquid includes ethanol and the terpenoid compound is dispersed in the ethanol.

3. The composition of claim 1, wherein the agent includes a compound of plant origin.

4. The composition of claim 3, wherein the compound includes camphor of plant origin.

5. The composition of claim 3, wherein the compound includes carvacrol of plant origin.

6. The composition of claim 3, wherein the compound includes thymol of plant origin.

7. The composition of claim 3, wherein the compound includes incensole acetate of plant origin.

8. The composition of claim 1, wherein the agent includes a compound of synthetic origin.

9. The composition of claim 8, wherein the compound includes camphor of synthetic origin.

10. The composition of claim 8, wherein the compound includes carvacrol of synthetic origin.

11. The composition of claim 8, wherein the compound includes thymol of synthetic origin.

12. The composition of claim 8, wherein the compound includes incensole of synthetic origin.

13. The composition of claim 1, wherein the liquid includes a second agent that activates a TRPA1 channel.

14. The composition of claim 13, wherein the second agent includes 6-paradol.

15. The composition of claim 13, wherein the second agent includes cinnamon aldehyde.

16. The composition of claim 1, wherein the liquid includes: approximately up to 15% extract of aframomum melegueta, up to approximately 15% extract of Cinnamomum Verum, up to approximately 70% extract of Thymus vulgaris, and up to approximately 15% extract of rosmarinus officinalis.

17. A device, comprising:
a liquid that aerosolizes for delivery to a user by an airway, the liquid including an agent that activates a TRPV3 channel, and the agent including a terpenoid compound; and
a housing that delivers the liquid aerosolized to the user.

18. The device of claim 17, wherein the housing is sized and shaped to mimic a cigarette.

19. The device of claim 18, wherein the housing is provided by one or more components of an electronic cigarette.

20. The device of claim 18, wherein the housing includes a porous medium and the liquid is disposed in the porous medium.

21. The device of claim 17, wherein the housing is provided by one or more components of an inhaler.

22. The device of claim 17, wherein the terpenoid compound includes one or more of camphor, carvacrol, thymol and incensole of either a plant or synthetic origin.

23. The device of claim 17, wherein the liquid includes a second agent that activates a TRPA1 channel.

24. The device of claim 23, wherein the second agent includes one or more of 6-paradol and cinnamon aldehyde.

25. The device of claim 17, wherein the liquid includes: approximately up to 15% extract of aframomum melegueta, approximately up to 15% extract of Cinnamomum Verum, approximately up to 70% extract of Thymus vulgaris, and approximately up to 15% extract of rosmarinus officinalis.

26. A method, comprising:
administering an agent that is an activator of a TRPV3 channel as an aerosol to a respiratory tract of a subject, the agent including a terpenoid compound.

27. The method of claim 26, the terpenoid compound includes one or more of camphor, carvacrol, thymol and incensole of either a plant or synthetic origin.

28. The method of claim 26, further comprising administering, with the agent, a second agent that is an activator of a TRPA1 channel.

29. The method of claim 28, wherein the second compound agent one or more of 6-paradol and cinnamon aldehyde.

30. The method of claim 26, wherein the administering includes administering the agent to the subject experiencing cigarette craving to reduce cigarette craving experienced by the subject.

31. The method of claim 26, wherein the administering includes administering the agent to the subject to mimic smoking.

32. The method of claim 26, wherein the administering includes administering the agent to the subject experiencing anxiety to reduce anxiety experienced by the subject.

33. The method of claim 32, wherein the administering includes administering the agent to the subject experiencing the anxiety from smoking withdrawal to reduce the anxiety experienced by the subject.
